SEQUENCE LISTING (30 sequences)

SEQ ID NO: 1 - protein, 230 aa, Artificial Sequence
  synthetic invariant amino acid residues between subtypes H1N1, H3N2 and H5N1 in the NS1 protein
MXXXXXXXFQVXCFLWXXRKXXXXXXXXDXPFXDRXXRXXXXXXGRXXTXXXXIXXXXXXGXXIXXXXXXXXXXXXXXXXXXSXXXXXYXXXMXXXXXXXXXXXXXXXXXXXXXXXXXXDQXXXXKXXXLXXXXXXXXXXXXXXXLXRXXXXXXXXXXGXXXXXXXXXXXXXXXXXXXXXXXXXXXEXXXXXXXXXXXXXXXXXXXXXXEXXXXXXXXXXXXXXXXXXXX

SEQ ID NO: 2 - protein, 4 aa, Artificial Sequence
  synthetic amino acid sequence for representative PL motif of Influenza A NS1 protein
ESEV

SEQ ID NO: 3 - protein, 4 aa, Artificial Sequence
  synthetic amino acid sequence for representative PL motif of Influenza A NS1 protein
ESEI

SEQ ID NO: 4 - protein, 4 aa, Artificial Sequence
  synthetic amino acid sequence for representative PL motif of Influenza A NS1 protein
ESKV

SEQ ID NO: 5 - protein, 4 aa, Artificial Sequence
  synthetic amino acid sequence for representative PL motif of Influenza A NS1 protein
TSEV

SEQ ID NO: 6 - protein, 4 aa, Artificial Sequence
  synthetic amino acid sequence for representative PL motif of Influenza A NS1 protein
GSEV

SEQ ID NO: 7 - protein, 4 aa, Artificial Sequence
  synthetic amino acid sequence for representative PL motif of Influenza A NS1 protein
RSEV

SEQ ID NO: 8 - protein, 4 aa, Artificial Sequence
  synthetic amino acid sequence for representative PL motif of Influenza A NS1 protein
RSKV

SEQ ID NO: 9 - protein, 4 aa, Artificial Sequence
  synthetic amino acid sequence for representative PL motif of Influenza A NS1 protein
GSEI

SEQ ID NO: 10 - protein, 4 aa, Artificial Sequence
  synthetic amino acid sequence for representative PL motif of Influenza A NS1 protein
GSKV

SEQ ID NO: 11 - protein, 4 aa, Artificial Sequence
  synthetic amino acid sequence for representative PL motif of Influenza A NS1 protein
NICI

SEQ ID NO: 12 - protein, 4 aa, Artificial Sequence
  synthetic amino acid sequence for representative PL motif of Influenza A NS1 protein
TICI

SEQ ID NO: 13 - protein, 4 aa, Artificial Sequence
  synthetic amino acid sequence for representative PL motif of Influenza A NS1 protein
RICI

SEQ ID NO: 14 - protein, 4 aa, Artificial Sequence
  synthetic amino acid sequence for representative PL motif of Influenza A NS1 protein
DMAL

SEQ ID NO: 15 - protein, 4 aa, Artificial Sequence
  synthetic amino acid sequence for representative PL motif of Influenza A NS1 protein
DMTL

SEQ ID NO: 16 - protein, 4 aa, Artificial Sequence
  synthetic amino acid sequence for representative PL motif of Influenza A NS1 protein
DIAL

SEQ ID NO: 17 - protein, 4 aa, Artificial Sequence
  synthetic amino acid sequence for representative PL motif of Influenza A NS1 protein
DLDY

SEQ ID NO: 18 - protein, 4 aa, Artificial Sequence
  synthetic amino acid sequence for representative PL motif of Influenza A NS1 protein
SICL

SEQ ID NO: 19 - protein, 233 aa, Artificial Sequence
  synthetic amino acid consensus sequence of NS1 protein from subtypes H1N1, H3N2, and H5N1
MDSNTVLSFQVDCFLWHVRKRFADQELGDAPFLDRLRRDQKSLRGRGNTLGLDIETATRAGKQIVERILEEESDEALKMTIASVPASRYLTDMTLEEMSRDWFMLMPKQKVAGSLCIKMDAIMDKTIILKANFSVIFDRLETLILLRAFTEEGAIRVGEISPLPSLPGHTGEDVKNAIGVLIGGLEWNDNTVRVSENTIQRFAWRGSDEDGRLPFPPNQKRKMARTIESEVEK

SEQ ID NO: 20 - protein, 10 aa, Artificial Sequence
  synthetic amino acid sequence of HA0 cleavage site in virulent H5 and H7 strains of influenza A
PEIPKGRGLF

SEQ ID NO: 21 - protein, 10 aa, Artificial Sequence
  synthetic amino acid sequence of HA0 cleavage site in virulent H5 and H7 strains of influenza A
PENPKGRGLF

SEQ ID NO: 22 - protein, 12 aa, Artificial Sequence
  synthetic amino acid sequence of HA0 cleavage site in virulent H5 and H7 strains of influenza A
PEIPKKKKRGLF

SEQ ID NO: 23 - protein, 14 aa, Artificial Sequence
  synthetic amino acid sequence of HA0 cleavage site in virulent H5 and H7 strains of influenza A
PETPKRKRKRGLSF

SEQ ID NO: 24 - protein, 13 aa, Artificial Sequence
  synthetic amino acid sequence of HA0 cleavage site in virulent H5 and H7 strains of influenza A
PEIPKKREKRGLF

SEQ ID NO: 25 - protein, 12 aa, Artificial Sequence
  synthetic amino acid sequence of HA0 cleavage site in virulent H5 and H7 strains of influenza A
PETPKRRRRGLF

SEQ ID NO: 26 - protein, 4 aa, Artificial Sequence
  synthetic amino acid sequence of a non-natural PDZ domain
GLGF

SEQ ID NO: 27 - protein, 219 aa, Influenza A virus
MDSNTMSSFQVDCFLWHIRKRFADNGLGDAPFLDRLRRDQKSLKGRGNTLGLDIETATLVGKQIVEWILKEESSETLRMTIASVPTSRYLSDMTLEEMSRDWFMLMPRQKIIGPLCVRLDQAIMEKNIVLKANFSVIFNRLETLILLRAFTEEGAIVGEISPLPSLPGHTYEDVKNAVGVLIGGLEWNGNTVRVSENIQRFAWRNCDENGRPSLPPEQK

SEQ ID NO: 28 - protein, 230 aa, Influenza A virus strain A/Wilson-Smith/1933 H1N1
MDPNTVSSFQVDCFLWHVRKRVADQELGDAPFLDRLRRDQKSLRGRGSTLGLDIETATRAGKQIVERILKEESDEALKMTMASVPASRYLTDMTLEEMSRHWFMLMPKQKVAGPLCIRMDQAIMDKNIILKANFSVILDRLETLILLRAFTEEGTIVGEISPLPSLPGHTDEDVKNAVGVLIGGLEWNNNTVRVSETLQRFAWRSSNENGRPPLTPKQKRKMAGTIRSEV

SEQ ID NO: 29 - protein, 230 aa, Influenza A virus strain A/Beijing/352/1989 H3N2
MDSNTVSSFQVDCFLWHIRKQVVDQELSDAPFLDRLRRDQRSLRGRGNTLGLDIKAATHVGKQIVEKILKEESDEALKMTMASTPASRYITDMTIEELSRNWFMLMPKQKVEGPLCIRMDQAIMEKNIMLKANFSVIFDRLETLVLLRAFTEEGAIVGEISPLPSFPGHTIEDVKNAIGVLIGGLEWNDNTVRVSKNLQRFAWGSSNENGGPPLTPKQKRKMARTARSKV

SEQ ID NO: 30 - protein, 230 aa, Influenza A virus strain A/Chicken/Scotland/19591
MDSNTVSSFQVDCFLWHVRKRFADQELGDAPFLDRLRRDQKSLRGRGSTLGLDIETATRAGKQIVERILEEESDEALKMTIASVPASRYLTDMTLEEMSRDWFMLMPKQKVAGSLCIRMDQAIMDKNIILKANFSVISDRLETLILLRAFTEEGAIVGEISPLPSLPGHTDEDVKNAIGVLIGGLEWNDNTVRVSETLQRFAWRSSNEDGRPPLPPKQKRKMARTIESEV

(19) United States
(12) Patent Application Publication
(10) Pub. No.: US 2011/0027775 A1
(43) Pub. Date: Feb. 3, 2011

(54) DETECTION OF INFLUENZA VIRUS

(75) Inventors: Peter S. Lu, Palo Alto, CA (US); Michael P. Belmares, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Arbor Vita Corporation, Sunnyvale, CA (US)

(21) Appl. No.: 12/840,468

(22) Filed: Jul. 21, 2010

Related U.S. Application Data
(60) Provisional application No. 61/227,424, filed on Jul. 21, 2009.

(51) Int. Cl. C12Q 1/70

(52) U.S. Cl. 435/5

(57) ABSTRACT

A change in strain of flu and consequently pandemic potential can be determined by assessing the presence or absence of a PL motif. The 2009 swine flu illustrates the utility of such a test. The swine flu is a subtype H1N1 influenza A. Swine flu differs from the vast majority of influenza H1N1 subtype strains from 1981-2008 or H3N2 strains from 1985 to the present in that its NS1 protein lacks a PL motif. PDZ polypeptides can be used to identify such strains and distinguish them from strains in which PL motifs are present.

### CROSS-REFERENCES TO RELATED APPLICATIONS

This application claims the benefit under 35 U.S.C. §1.119(e) of U.S. Application No. 61/227,424, filed Jul. 21, 2009, which is incorporated by reference in its entirety for all purposes.

### BACKGROUND OF THE INVENTION

Influenza is caused by an RNA virus of the orthomyxoviridae family. There are three types of these viruses and they cause three different types of influenza: type A, B and C. Influenza virus type A viruses infect mammals (humans, pigs, ferrets, horses) and birds. This type of virus has caused worldwide pandemics. Influenza virus type B (also known simply as influenza B) infects only humans. It occasionally causes local outbreaks of flu. Influenza type C viruses also infect only humans. They infect most people when they are young and rarely causes serious illness.

New strains of influenza A, some with pandemic potential, can emerge rapidly at any time. An example is the 2009 pandemic of swine flu, also known as A 2009 (H1N1). The ability to identify new strains of influenza A rapidly to permit early therapeutic intervention and/or quarantining of infected subjects is important in preventing developments of pandemics. Unfortunately, A 2009 (H1N1) was not identified in sufficient time to prevent it developing into a pandemic.

Current rapid immunodiagnostic tests for influenza antigens like “Binax NOW FluA and FluB™” (Binax, Inc., Portland, Me.), “Directigen Flu A+B™” (Becton Dickinson, Franklin Lakes, N.J.), “Flu OIA™” (Biostar Inc., Boulder, Colo.), “Quick Vue™” (Quidel, Sand Diego, Calif.), “Influ AB Quick™” (Denka Sieken Co., Ltd., Japan) and “Xpect Flu A & B” (Remel Inc., Lenexa, Kans.), can reportedly either detect influenza A or distinguish between influenza A and B. The complexity of the test formats may require special training. In addition, significant amounts of virion particles are commonly required to obtain a positive test result, limiting their use to a short window of time when virus shedding is at its highest levels. These assays are not capable of subtyping (e.g., distinguishing H5N1 from H1N1) or distinguishing strains within a subtype.

Reverse-transcriptase PCR-based diagnostics (RT-PCR) has resulted in advances in capabilities, but is laborious and requires highly trained personnel making on-site or field-testing difficult. Because of the relative inefficiency of the reverse transcriptase enzyme, significant amounts of virus (e.g., 10⁴ virion particles) and as many as 20 primers may be required effectively to detect viral RNA. RT PCR is not easily adapted to high throughput screening of subjects in an epidemic setting or to field uses in an agricultural or point-of-care setting. RT-PCR is capable of distinguishing subtypes for which prior sequence information is available but not newly emergent strains.

### BRIEF SUMMARY OF THE INVENTION

The invention provides methods of analyzing a sample for influenza A infection. The methods comprise contacting a sample with a pan-specific binding reagent for an influenza A protein and with a PL-motif binding reagent; determining binding of the pan-specific binding reagent and the PL-motif binding agent to the sample; and analyzing the sample from comparative binding of the pan-specific binding reagent and the PL-motif binding agent, wherein (a) detectable binding of the pan-specific binding agent and lack of detectable binding of the PL-motif binding agent provides an indication the sample contains a strain of influenza A lacking a PL-motif; (b) detectable binding of the pan-specific binding agent and the PL-motif binding agent provides an indication the sample contains a strain of influenza A containing a PL motif; and (c) lack of detectable binding of both the pan-specific binding agent and the PL-motif binding agent provides an indication the sample is not infected with influenza A. In some methods, the determining step indicates detectable binding of the pan-specific binding agent and lack of detectable binding of the PL-motif binding agent providing an indication the sample contains a strain of influenza A lacking a PL motif. Some methods further comprise informing a subject from whom the sample was obtained or a physician treating the patient that the method has provided an indication the sample contains a strain of influenza A lacking a PL motif. Some methods further comprise subjecting the subject from whom the sample was obtained to a new treatment regime or quarantine regime. In some methods, the pan-specific binding reagent is an antibody that specifically binds to an influenza A NS1 protein. In some methods, the pan-specific binding reagent is a monoclonal antibody that specifically binds to an influenza A NS1 protein.

In some methods, the PL motif binding reagent is a first PDZ polypeptide and the contacting step further comprises contacting the sample with a second PDZ polypeptide, and the determining step further comprises determining binding of the second PDZ polypeptide to the sample; wherein greater binding of the pan-specific binding reagent that either the first or second PDZ polypeptides provides an indication that the sample contains a strain of influenza A lacking a PL motif. In some methods, (a) detectable binding of the pan-specific binding agent and lack of detectable binding of the first and second PL-motif binding agents provides an indication the sample contains a strain of influenza A lacking a PL motif; (b) detectable binding of the pan-specific binding agent and either or both of the PL-motif binding agent provides an indication the sample contains a strain of influenza A containing a PL motif; and (c) lack of detectable binding of both the pan-specific binding agent and the PL-motif binding agents provides an indication the sample is not infected with influenza A. In some methods, the determining step indicates detectable binding of the pan-specific binding agent and lack of detectable binding of the first and second PL-motif binding agents and provides an indication the sample contains a strain of influenza A lacking a PL motif. In some methods, the pan-specific binding reagent is an antibody that specifically binds to an influenza A NS1 protein, the first PDZ polypeptide comprises INADL domain 8 and the second PDZ polypeptide comprises a PSD95 PDZ domain. In some methods, the second PDZ polypeptide comprises PSD95 PDZ domains 1, 2 and 3. In some methods, the second PDZ polypeptide comprises three copies of PDZ domain 3.

In some methods, the determining step is performed by sandwich assays. In some methods, the pan-specific binding reagent and the PL-motif binding reagent are capture agents and a second pan-specific binding reagent is detection agent in the sandwich assay. In some methods, the pan-specific binding reagent is a polyclonal antibody to NS1, the second pan-specific binding reagent is a polyclonal antibody to NS1, and the PL-motif binding agent is a PDZ polypeptide. In some methods, the same detection agent is used in determining binding of the pan-specific binding reagent and the PL-motif binding agent. In some methods, the pan-specific binding agent is a monoclonal antibody to NS1, the second pan-specific binding agent is a second monoclonal antibody to NS1 binding to a different epitope that the monoclonal antibody to NS1, the PL-motif binding agent is a PDZ polypeptide, and the second monoclonal antibody is used as the detection agent in determining binding of the pan-specific binding reagent and the PL-motif binding agent. In some methods, the sample is from an individual suspected of being infected with a strain of influenza A lacking a PL motif.

In some methods, the sample is from an individual exposed to another subject having influenza A lacking a PL motif. In some methods, the sample is from an individual suspected of being infected with H1N1 influenza A lacking a PL motif. In some methods, the sample is from an individual suspected of being infected with A 2009 (H1N1).

The invention further provides methods of analyzing a sample for influenza A infection. Such methods comprise contacting a sample with a pan-specific binding reagent for an influenza A protein and with a PL-motif binding reagent; determining binding of the pan-specific binding reagent and the PL-motif binding agent to the sample; wherein the determining indicates detectable binding of the pan-specific binding agent and lack of detectable binding of the PL-motif binding agent and thereby provides an indication the sample contains a strain of influenza A lacking a PL motif.

The invention further provides methods of analyzing a sample suspected of containing strain of influenza A virus lacking a PL motif. Such methods comprise contacting a sample with a pan-specific binding reagent for an influenza A protein and with a PL-motif binding reagent; determining binding of the pan-specific binding reagent and the PL-motif binding agent to the sample; wherein (a) detectable binding of the pan-specific binding agent and lack of detectable binding of the PL-motif binding agent provides an indication the sample contains a strain of influenza A lacking a PL motif; (b) detectable binding of the pan-specific binding agent and the PL-motif binding agent provides an indication the sample contains a strain of influenza A containing a PL motif; and (c) lack of detectable binding of both the pan-specific binding agent and the PL-motif binding agent provides an indication the sample is not infected with influenza A. In some such methods, the sample is suspected of containing influenza A of H1N1 subtype lacking a PL motif. In some such methods, the sample is suspected of containing 2009 swine flu.

The invention further provides methods of analyzing a sample for a drug-resistant virus, comprising contacting a sample with an antiviral drug; and determining binding of the antiviral drug to the sample, wherein presence or extent of binding of the drug to the sample provides an indication that the sample contains a drug-sensitive virus. Some such methods further comprise contacting the sample with a binding agent that specifically binds to a virus and the determining step determines binding of the binding agent to the sample; wherein presence or extent of binding of the binding agent to the sample provides an indication the sample contains the virus. In some methods, the binding agent and drug specifically bind to the same protein of the virus. In some methods, the binding agent is a antibody to an NS1 protein of influenza virus. In some methods, the binding agent is an antibody to an NS1 protein of influenza virus A. In some methods, the binding agent is a polyclonal antibody. In some methods, the binding agent is a monoclonal antibody. In some methods, the drug is immobilized to a support. In some methods, the drug and binding agent are immobilized to the same support. In some methods, the drug and the binding agent are immobilized to the same support and form capture agents for determining of the binding of the drug and binding agent to the sample by sandwich assay. In some methods, the same detection agent is used for the drug and the binding agent and detection agent is an antibody. In some methods, a different detection agent is used for the drug and the binding agent. In some methods, the contacting step comprises containing the sample with a plurality of antiviral drugs. In some methods, the plurality of antiviral drugs are immobilized to the same support. In some methods, the drug is tamiflu or relenza. In some methods, the drug is amantadine or rimantadine. In some methods, the sample is suspected of containing influenza virus. In some methods, the sample is suspected of containing influenza virus A.

The invention further provides a method of analyzing a plurality of samples for influenza A infection, comprising contacting the plurality of sample with a pan-specific binding reagent for an influenza A protein and with a PL-motif binding reagent; determining binding of the pan-specific binding reagent and the PL-motif binding agent to the plurality of samples; analyzing the samples from comparative binding of the pan-specific binding reagent and the PL-motif binding agent, wherein (a) at least one sample shows detectable binding of the pan-specific binding agent and lack of detectable binding of the PL-motif binding agent providing an indication it contains a strain of influenza A lacking a PL-motif; and (b) at least one sample shows lack of detectable binding of both the pan-specific binding agent and the PL-motif binding agent providing an indication the sample is not infected with influenza A. In some such methods, at least one sample shows detectable binding of the pan-specific binding agent and the PL-motif binding agent providing an indication it contains a strain of influenza A containing a PL motif.

### BRIEF DESCRIPTION OF THE DRAWINGS

FIG. 1 shows invariant amino acids in the NS1 protein between H1N1, H3N2 and H5N1 subtypes.

FIG. 2 shows a consensus sequence of amino acids between H1N1, H3N2 and H5N1 strains in the NS1 protein.

FIG. 3 shows sequences of A 2009 (H1N1) (Swine flu) (A), which lacks a PL motif, compared with NS1 protein from exemplary H1N1 (B), H3N2 (C) and H5N1 (D) strains each of which contains a PL motif.

FIG. 4 is a schematic lateral flow assay to identify Swine flu. Three binding reagents are deposited on the strip: goat anti-mouse antibody (C) (positive control), pan-specific anti-NS1 antibody (L1) and INADL domain 8(L2) A pattern containing three bands indicates presence of seasonal flu A, whereas presence of the C and L1 stripes and not the L2 bands indicates swine flu.

FIG. 5 illustrates expansion of the previous assay by including two PDZ polypeptides (L2 and L3) to distinguish seasonal flu A and H5N1 avian flu as well as identifying swine flu. PSD95 is suitable for use as the L2 PDZ polypeptide and INADL d8 as the L3 PDZ polypeptide.

FIG. 6 is a schematic illustrating use of strip test to type influenza A for drug resistance. Two common influenza drugs, tamiflu and Relenza are deposited on a strip together with goat anti-mouse antibody (C) as a positive control, and a pan-specific antibody for NS1 (L1) to indicate presence of influenza A. Bands for L2 and L3 indicate sensitivity to these drugs.

### DEFINITIONS

“Avian influenza A” means an influenza A subtype that infects an avian subject and is transmissible between avian subjects. Representative examples of avian influenza hemagglutinin subtypes include H5, H6, H7, H9 and H10 and representative strains include H5N1, H6N2, H7N3, H7N7, H9N2, H10N4 and H10N5. Some strains of Avian influenza can also infect humans.

“Avian subject” means a subject suitable for testing or treatment including all species of birds, including both wild birds (such as wildfowl) and domesticated species (such as poultry). Subject include chickens, turkeys, ducks, geese, quail, ostrich, emus and exotic birds such as parrots, cockatoos and cockatiels.

“Pathogenic strain of influenza A” when used in the context of distinguishing between different strains of influenza virus means a “notifiable avian influenza” (NAI) virus according to the guidelines set forth by the OIE World Organization for Animal Health, World Health Organization or their designated representatives e.g., as set forth in the OIE “Manual of Diagnostic Tests and Vaccines for Terrestrial Animals, 5th edition, 2004 (www.oie.int). Further, the subject pathogenic strain has “high pathogenicity” in a representative test for virulence or an H5 or H7 virus with an influenza A hemagglutinin (HA) precursor protein HA0 cleavage site amino acid sequence that is similar to any of those that have been observed in virulent viruses, i.e., as defined by the OIE or a representative similar national or international organization or trade association. Representative examples of HA0 cleavage site amino acid sequences in virulent H5 and H7 strains of influenza A comprise multiple basic amino acids (arginine or lysine) at the cleavage site of the viral precursor hemagglutinin protein, e.g., where low virulence strains of H7 viruses have PEIPKGR*GLF (SEQ ID NO:20) or PENPKGR*GLF (SEQ ID NO:21) highly pathogenic strains have -PEIPKKKKR*GLF (SEQ ID NO:22), PETPKRKRKR*GLSF (SEQ ID NO:23), PEIPKKREKR*GLF (SEQ ID NO:24) or PETPKRRRR*GLF (SEQ ID NO:25). Current representative tests for virulence include inoculation of 4-8 week old chickens with infectious virus wherein strains are considered to be highly pathogenic if they cause more than 75% mortality within 10 days; and/or, any virus that has an intravenous pathogenicity index (IVPI) greater than 1.2, wherein intravenously inoculated birds are examined at 24-hour intervals over a 10-day period; scored for “0”, normal; “1” sick; “2” severely sick”; “3” dead; and, the mean score calculated as the IVPI. The latter highly pathogenic strains are referred to by the OIE as a “highly pathogenic NAI virus” (HPNIA). Current representative examples of NAI include the H5 and H7 strains of influenza A. Current representative examples of HPNIA include H5N1.

“Less Pathogenic strain of influenza A” means an avian influenza A that is notifiable, i.e., an NAI isolate (supra), but which is not pathogenic for chickens and does not have an HA0 cleavage site amino acid sequence similar to any of those that have been observed in virulent viruses, e.g., a strain referred to by the OIE as a “low pathogenicity avian influenza (LPAI).

Strains of influenza A that are not classified as highly pathogenic or less pathogenic (i.e., are not notifiable) are referred to as seasonal flu. Most strains of influenza A H1N1 and H3N2 are seasonal flu. However, one strain responsible for the 1918 Spanish flu is highly pathogenic.

“PDZ domain” means an amino acid sequence having at least 50, 60, 70, 80 or 90% sequence identity with a PDZ domain from at least one of brain synaptic protein PSD95, the Drosophila septate junction protein Discs-Large (DLG) and/or the epithelial tight junction protein ZO1 (ZO1), and animal homologs. Sequence identities of PDZ domains are determined over at least 70 amino acids within the PDZ domain, preferably 80 amino acids, and more preferably 80-90 or 80-100 amino acids. Amino acids of analogs are assigned the same numbers as corresponding amino acids in the natural human sequence when the analog and human sequence are maximally aligned. Analogs typically differ from naturally occurring peptides at one, two or a few positions, often by virtue of conservative substitutions. Representative examples of PDZ proteins include CASK, MPP1, DLG1, DLG2, PSD95, NeDLG, TIP-33, TIP-43, LDP, LIM, LIMK1, LIMK2, MPP2, AF6, GORASP1, INADL, KIAA0316, KIAA1284, MAGI1, MAST2, MINT1, NSP, NOS1, PAR3, PAR3L, PAR6 beta, PICK1, Shank 1, Shank 2, Shank 3, SITAC-18, TIP1, and ZO-1. PDZ domains can be naturally occurring or non-naturally occurring. Representative examples of PDZ domains include allelic variants of PDZ proteins, as well as, chimeric PDZ domains containing portions of two different PDZ proteins and the like. Representative non-natural PDZ domains include those in which the corresponding genetic code for the amino acid sequence has been mutated, e.g., to produce amino acid changes that alter (strengthen or weaken) either binding or specificity of binding to PL. The term “allelic variant” is used to refer to variations between genes of different individuals in the same species and corresponding variations in proteins encoded by the genes. An exemplary PDZ domain for PSD95 d2 is described in WO 08/094,525.

PDZ polypeptide means a naturally occurring or non-naturally occurring protein including a PDZ domain as described above. Some PDZ polypeptide include a PDZ domain fused to a tag to facilitate detection or immobilization (e.g., glutathione S-transferase or GST, myc, hexa-histidine or FLAG. Some PDZ polypeptides contain a PDZ domain that is smaller than a natural PDZ domain. For example a non-natural PDZ domain may optionally contain a “GLGF” motif, i.e., a motif having the GLGF amino acid sequence (SEQ ID NO:26), which typically resides proximal, e.g. usually within about 10-20 amino acids N-terminal, to an PDZ domain. The latter GLGF motif (SEQ ID NO:26), and the 3 amino acids immediately N-terminal to the GLGF motif (SEQ ID NO:26) are often required for PDZ binding activity. Similarly, non-natural PDZ domains may be constructed that lack the β-sheet at the C-terminus of a PDZ domain, i.e., this region may often be deleted from the natural PDZ domain without affecting the binding of a PL. Some exemplary PDZ proteins are provided and the GI or accession numbers are provided in parenthesis: PSMD9 (9184389), af6 (430993), AIPC (12751451), ALP (2773059), APXL-1 (13651263), MAGI2 (2947231), CARDI1 (1282772), CARDI4 (13129123), CASK (3087815), CNK1 (3930780), CBP (3192908), Densin 180 (16755892), DLG1 (475816), DLG2 (12736552), DLG5 (3650451), DLG6 splice var 1 (14647140), DLG6 splice var 2 (AB053303), DVL1 (2291005), DVL2 (2291007), DVL3 (6806886), ELFIN 1 (2957144), ENIGMA (561636), ERBIN (8923908), EZRIN binding protein 50 (3220018), FLJ00011 (10440342), FLJ11215 (11436365), FLJ12428 (BC012040), FLJ12615 (10434209), FLJ20075 Semcap2 (7019938), FLJ21687 (10437836), FLJ31349 (AK055911), FLJ32798 (AK057360), GoRASP1 (NM031899), GoRASP2 (13994253), GRIP1 (4539083), GTPase Activating Enzyme (2389008), Guanine Exchange Factor (6650765), HEMBA 1000505 (10436367), HEMBA 1003117 (7022001), HSPC227 (7106843), HTRA3 (AY040094), HTRA4 (AL576444), INADL (2370148), KIAA0147 Vartul (1469875), KIAA0303 MAST4 (2224546), KIAA0313 (7657260), KIAA0316 (6683123), KIAA0340 (2224620), KIAA0380 (2224700), KIAA0382 (7662087), KIAA0440 (2662160), KIAA0545 (14762850), KIAA0559 (3043641), KIAA0561 MAST3 (3043645), KIAA0613 (3327039), KIAA0751 RIM2 (12734165), KIAA0807 MAST2 (3882334), KIAA0858 (4240204), KIAA0902 (4240292), KIAA0967 (4589577), KIAA0973 SEMCAP3 (5889526), KIAA1202 (6330421), KIAA1222 (6330610), KIAA1284 (6331369), KIAA1389 (7243158), KIAA1415 (7243210), KIAA1526 (5817166), KIAA1620 (10047316), KIAA1634 MAGI3 (10047344), KIAA1719 (1267982), LIM Mystique (12734250), LIM (3108092), LIMK1 (4587498), LIMK2 (1805593), LIM-RIL (1085021), LU-1 (U52111), MAGI1 (3370997), MGC5395 (BC012477), MINT1 (2625024), MINT3 (3169808) MPP1 (189785), MPP2 (939884), MPP3 (1022812), MUPP1 (2104784), NeDLG (10853920), Neurabin II (AJ401189), NOS1 (642525), novel PDZ gene (7228177), Novel Serine Protease (1621243), Numb Binding Protein (AK056823), Outer Membrane Protein (7023825), p55T (12733367), PAR3 (8037914), PAR3-like (AF428250), PAR6 (2613011), PAR6BETA (13537116), PAR6GAMMA (13537118), PDZ-73 (5031978), PDZK1 (2944188), PICK1 (4678411), PIST (98394330), prIL16 (1478492), PSAP (6409315), PSD95 (3318652), PTN-3 (179912), PTN-4 (190747), PTPL1 (515030), RGS12 (3290015), RGS3 (18644735), Rho-GAP10 (NM020824), Rhophilin-like (14279408), Serine Protease (2738914), Shank 2 (6049185), Shank 3 (AC000036), Shroom (18652858), Similar to GRASP65 (14286261), Similar to Ligand of Numb px2 (BC036755), Similar to PTP Homolog (21595065), SIP1 (2047327), SITAC-18 (8886071), SNPCIIA (20809633), Shank 1 (7025450), Syntenin (2795862), Syntrophin 1 alpha (1145727), Syntrophin beta 2 (476700), Syntrophin gamma 1 (9507162), Syntrophin gamma 2 (9507164), TAX2-like protein (3253116), TIAM 1 (4507500), TIAM 2 (6912703), TIP 1 (2613001), TIP2 (2613003), TIP33 (2613007), TIP43(2613011), X-11 beta (3005559), ZO-1 (292937), ZO-2 (12734763), ZO-3 (10092690).

“PDZ ligand,” abbreviated “PL”, means a naturally occurring protein that includes an amino acid sequence (PL motif) which binds to and forms a molecular interaction complex with a PDZ-domain. In the case of NS1 proteins of influenza A, PL motifs are usually characterized by the four amino acids at the C-terminus of the NS1 protein. Representative motifs are ESEV (SEQ ID NO:2), ESEI (SEQ ID NO:3), ESKV (SEQ ID NO:4), TSEV (SEQ ID NO:5), GSEV (SEQ ID NO:6), RSEV (SEQ ID NO:7), RSKV (SEQ ID NO:8) GSEI (SEQ ID NO:9), GSKV (SEQ ID NO:10), NICI (SEQ ID NO:11), TICI (SEQ ID NO:12), RICI (SEQ ID NO:13), DMAL (SEQ ID NO:14), DMTL (SEQ ID NO:15), DIAL (SEQ ID NO:16), DLDY (SEQ ID NO:17), SICL (SEQ ID NO:18), SEV, SEI, SKV, and SKI. Other exemplary motifs have been described in WO 07/018,843. A peptide include a PL motif is referred to as a PL peptide. A PL peptide can include at least 2, 3, 4, 5, 6, 7, 8, 9 contiguous amino acids from the C-terminus of a PL protein but usually includes no more than 10, 15 or 20 such amino acids.

An influenza virus NS1 protein lacking a PL motif means an NS1 protein lacking one of the above motifs and/or lacking detectable binding to at least PSD95 and INADL d8 PZA polypeptides and preferably all tested PDZ polypeptides. The length of an NS1 proteins also provides an indication whether a PL motif is present. Fewer than 225 amino acids and particularly fewer than 220 amino acids in length is an indication that no PL motif is present.

“Specific binding” between a binding agent, e.g., an antibody or a PDZ domain and a particular viral analyte, such as an NS1 protein, refers to the ability of the binding agent to preferentially bind to a particular viral analyte that is present in a mixture of different viral analytes (e.g., for an antibody to bind to NS1 from influenza A without specifically binding to NS1 from influenza B, or vice versa or for a PDZ domain to bind to a first PL motif without binding to a second PL motif or without binding to a protein lacking a PL motif). Specific binding usually means a dissociation constant (KD) that is less than about 10⁻⁶M; preferably, less than about 10⁻⁷M; and, most preferably, less than about 10⁻⁸ M, In some methods, specific binding interaction is capable of discriminating between proteins having or lacking a PL with a discriminatory capacity greater than about 10- to about 100-fold; and, preferably greater than about 1000- to about 10,000-fold.

“Capture agent/analyte complex” is a complex that results from the specific binding of a capture agent, with an analyte, e.g. an influenza viral NS1 protein. A capture agent and an analyte specifically bind, i.e., the one to the other, under conditions suitable for specific binding, wherein such physicochemical conditions are conveniently expressed e.g. in terms of salt concentration, pH, detergent concentration, protein concentration, temperature and time. The subject conditions are suitable to allow binding to occur e.g. in a solution; or alternatively, where one of the binding members is immobilized on a solid phase. Representative conditions so-suitable are described in e.g., Harlow and Lane, “Antibodies: A Laboratory Manual”, Cold Spring Harbor Laboratory, Cold Spring Harbor, N.Y. (1989). Suitable conditions preferably result in binding interactions having dissociation constants (KD) that are less than about 10⁻⁶M; preferably, less than about 10⁻⁷M; and, most preferably less than about 10⁻⁸M.

“Solid phase” means a surface to which one or more reactants may be attached electrostatically, hydrophobically, or covalently. Representative solid phases include e.g.: nylon 6; nylon 66; polystyrene; latex beads; magnetic beads; glass beads; polyethylene; polypropylene; polybutylene; butadiene-styrene copolymers; silastic rubber; polyesters; polyamides; cellulose and derivatives; acrylates; methacrylates; polyvinyl; vinyl chloride; polyvinyl chloride; polyvinyl fluoride; copolymers of polystyrene; silica gel; silica wafers glass; agarose; dextrans; liposomes; insoluble protein metals; and, nitrocellulose. Representative solid phases include those formed as beads, tubes, strips, disks, filter papers, plates and the like. Filters may serve to capture analyte e.g. as a filtrate, or act by entrapment, or act by covalently binding. A solid phase capture reagent for distribution to a user may consist of a solid phase coated with a “capture reagent”, and packaged (e.g., under a nitrogen atmosphere) to preserve and/or maximize binding of the capture reagent to an influenza NS1 analyte in a biological sample.

Biological samples include tissue fluids, tissue sections, biological materials carried in the air or in water and collected there from e.g. by filtration, centrifugation and the like, e.g., for assessing bioterror threats and the like. Alternative biological samples can be taken from fetus or egg, egg yolk, and amniotic fluids. Representative biological fluids include urine, blood, plasma, serum, cerebrospinal fluid, semen, lung lavage fluid, feces, sputum, mucus, water carrying biological materials and the like. Alternatively, biological samples include nasopharyngeal or oropharyngeal swabs, nasal lavage fluid, tissue from trachea, lungs, air sacs, intestine, spleen, kidney, brain, liver and heart, sputum, mucus, water carrying biological materials, cloacal swabs, sputum, nasal and oral mucus, and the like. Representative biological samples also include foodstuffs, e.g., samples of meats, processed foods, poultry, swine and the like. Biological samples also include contaminated solutions (e.g., food processing solutions and the like), swab samples from out-patient sites, hospitals, clinics, food preparation facilities (e.g., restaurants, slaughter houses, cold storage facilities, supermarket packaging and the like). Biological samples may also include in situ tissues and bodily fluids (i.e., samples not collected for testing), e.g., the instant methods may be useful in detecting the presence or severity or viral infection in the eye e.g., using eye drops, test strips applied directly to the conjunctiva; or, the presence or extent of lung infection by e.g. placing an indicator capsule in the mouth or nasopharynx of the test subject. Alternatively, a swab or test strip can be placed in the mouth. The biological sample may be derived from any tissue, organ or group of cells of the subject. In some embodiments a scrape, biopsy, or lavage is obtained from a subject. Biological samples may include bodily fluids such as blood, urine, sputum, and oral fluid; and samples such as nasal washes, swabs or aspirates, tracheal aspirates, chancre swabs, and stool samples. Samples can be collected for example as nasal swabs, washes or aspirates, or tracheal aspirates, oral swabs and the like.

The term “substantial identity” means that two peptide sequences, when optimally aligned, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over the comparison window, which is typically the entire length of a protein or sequence encoding a protein), share at least 65 percent sequence identity, preferably at least 80 or 90 percent sequence identity, more preferably at least 95 percent sequence identity or more (e.g., 99 percent sequence identity or higher). Preferably, residue positions which are not identical differ by conservative amino acid substitutions. The percentage sequence identity between the subject and reference sequences is the number of positions occupied by the same amino acid (or nucleotide) in both the subject and reference sequences divided by the total number of aligned positions of the two regions, with gaps not counted, multiplied by 100 to convert to percentage.

“Isolated” or “purified” generally refers to isolation of a substance (compound, polynucleotide, protein, polypeptide, polypeptide composition) such that the substance comprises a significant percent (e.g., greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample that is not found naturally.

“Subject”, is used herein to refer to a man and domesticated animals, e.g. mammals, fishes, birds, reptiles, amphibians and the like.

The present methods provide an indication whether or not influenza A or particular subtype or strain thereof is present in a sample. The present methods do not necessarily require ability to determine the presence or absence of influenza A, its subtypes and/or strains with 100% accuracy, rather to provide an increased probability that influenza A and/or a subtype and/or a strain is present in the subject compared to the probability before the diagnostic test was performed.

The epitope of a mAb is the region of its antigen to which the mAb binds. Two antibodies bind to the same or overlapping epitope if one competitively inhibits (blocks) binding of a prototypical antibody defining the competition group to the antigen (an NS1 protein of influenza A or influenza B, in the assays below). That is, a 3-fold of 5-fold excess of one antibody inhibits binding of the other by at least 50% but preferably 75%, 90% or even 99% as measured in a competitive binding assay compared to a control lacking the competing antibody (see, e.g., Junghans et al., Cancer Res. 50:1495, 1990, which is incorporated herein by reference). Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Detecting “presence” or “absence” of an analyte includes quantitative assays in which only presence or absence of analyte is detected and quantitative assays in which presence of analyte is detected as well as an amount of analyte present. An analyte is present in a sample if the measured signal indicating the analyte is greater beyond a margin of experimental error than the signal from a sample in which the analyte is known to be absent. Conversely, an analyte is absent if the signal from which presence of analyte would be inferred is the same as within a margin of experimental error as the signal from a sample in which the analyte is known to be absent. The margin of error can be assessed by any standard means (e.g., plus or minus one standard deviation of the mean of repeated measurements).

A sample is suspected of containing influenza A, or a subtype or strain of influenza A if the individual from who the sample was obtained has a higher probability of having the infection than other individuals in general of the same species. Such a higher probability may be, for example, the result of exposure to other subjects having the virus, a result of signs or symptoms of disease exhibited by the patient or of contact with swine.

### DETAILED DESCRIPTION OF THE INVENTION

### I. General

Commonly owned applications WO 08/094,525, WO 08/048,276 and WO 07/018,843 set out the general concept that NS1 protein of influenza A is an abundant protein in subjects infected with influenza A and is characterized by a C-terminal PL motif that differs subtypes of influenza A (e.g., avian influenza H5N1 and seasonal influenza H1N1 and H3N2). Accordingly, PDZ polypeptides can be used to detect influenza A and to distinguish between subtypes having different PL motifs.

The present application arises in part from the insight that the presence or absence of a PL motif in the predominant strains of H1N1 and H3N2 subtypes of influenza A has varied with time as shown below.

| | % strains with |
|---|---|
| | NS1 PL motif |
| H1N1 | |
| Pre-1946 | 95% |
| 1946-1981 | 2% |
| 1981-2008 | 99% |
| 2009 | 0% |
| H3N2 | |
| Pre-1985 | 3% |
| 1985-2002 | 97% |
| 2003- | 100% |

Thus, a change in strain of seasonal flu and consequently pandemic potential can be determined by assessing the presence or absence of a PL motif. The 2009 swine flu illustrates the utility of such a test. The swine flu is a subtype H1N1 influenza. Swine flu differs from the vast majority of influenza H1N1 subtype strains from 1981-2008 or H3N2 strains from 1985 to the present in that its NS1 protein lacks a PL motif. A PL motif has been present in substantially all H5N1 strains to date regardless of time period. PDZ polypeptides can be used to identify such strains and distinguish them from strains in which PL motifs are present.

A 2009 (H1N1) or other strains of influenza A lacking a PL motif can be identified from its inability to detectably bind one or more PDZ polypeptide. An exemplary assay includes a pan-specific antibody for influenza A virus NS1 and a PDZ polypeptide. Binding of the antibody provides an indication that influenza A is present in the sample. Lack of binding of the PDZ polypeptide provides an indication that the strain of influenza A lacks a PL motif. The present methods can readily be combined with the subtyping methods described in WO 08/094,525 by including two different PDZ polypeptides with binding specificities for different subtypes of influenza (e.g., PSD95 and INADL). In such assays, a pan-specific binding agent indicates whether the sample is infected with influenza A, and lack of binding to both PSD95 and INADL provides an indication that the samples contains a strain of influenza A lacking a PL motif.

The methods are useful both in identifying a newly emergent strain of influenza A that differs from immediately previous strains by the acquisition or loss of a PL motif and in detecting such strains after they have emerged as in the case for A 2009 (H1N1).

### II. Influenza Viruses and their NS1 Proteins

The influenza viruses belong to the Orthomyxoviridae family, and are classified into types A, B, and C based upon antigenic differences in their nucleoprotein (NP) and matrix protein (M1). Further subtyping into strains is commonly based upon assessing the type of antigen present in two virion glycoproteins, namely, hemagglutinin (HA; H) and neuraminidase (NA; N). HA and NP are virulence factors mediating attachment of the virion to the surface of host cells. Thus, H5N1, H1N1 and H3N2 are examples of subtypes of influenza A. Within each subtype there are hundreds of strains. M1 protein is thought to function in virus assembly and budding, whereas NP functions in RNA replication and transcription. In addition to these virion proteins, two other non-structural, i.e., non-virion, proteins are expressed in virus infected cells which are referred to as non-structural proteins 1 and 2 (NS1; NS2). The non-structural viral protein NS1 has multiple functions including the regulation of splicing and nuclear export of cellular mRNAs and stimulation of translation, as well as the counteracting of host interferon ability.

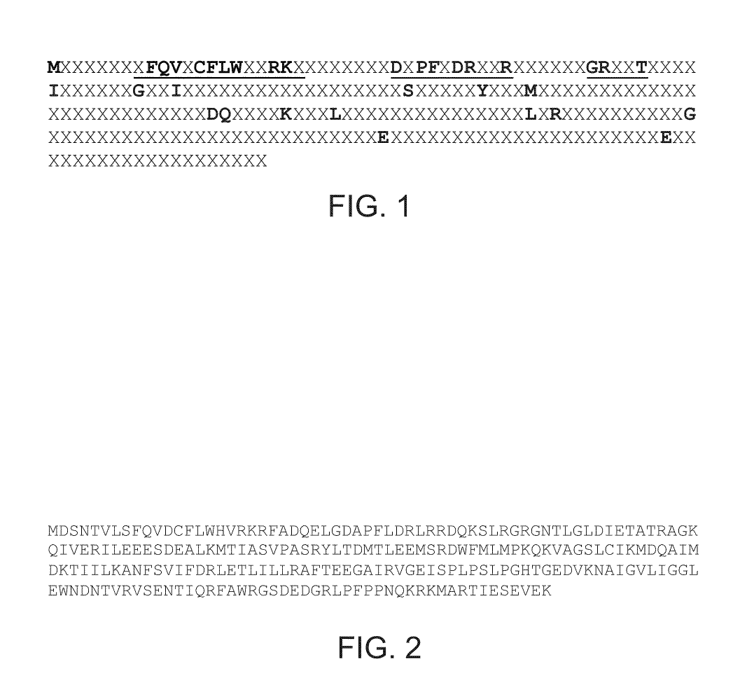

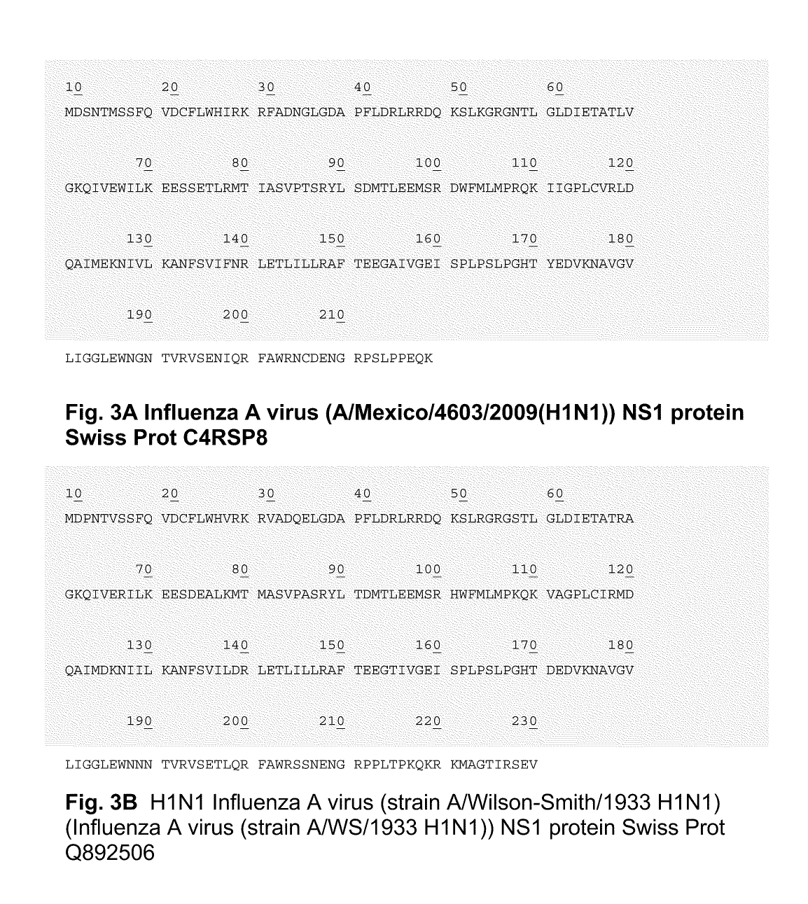

The NS1 protein has been identified and sequenced in influenza viruses and exemplary sequences can be found in the NCBI and Swiss-Prot database. The NS1 proteins from influenza A, B and C do not in general show antigenic cross reactivity. Within a type (e.g., influenza A), there is considerable variation in sequence between subtypes, but some antigenic crossreactivity depending on which antibody is used. FIG. 1 shows invariant residues between subtypes H1N1, H3N2 and H5N1 in the NS1 protein. FIG. 2 shows a consensus sequence of NS1 protein from subtypes H1N1, H3N2, and H5N1.

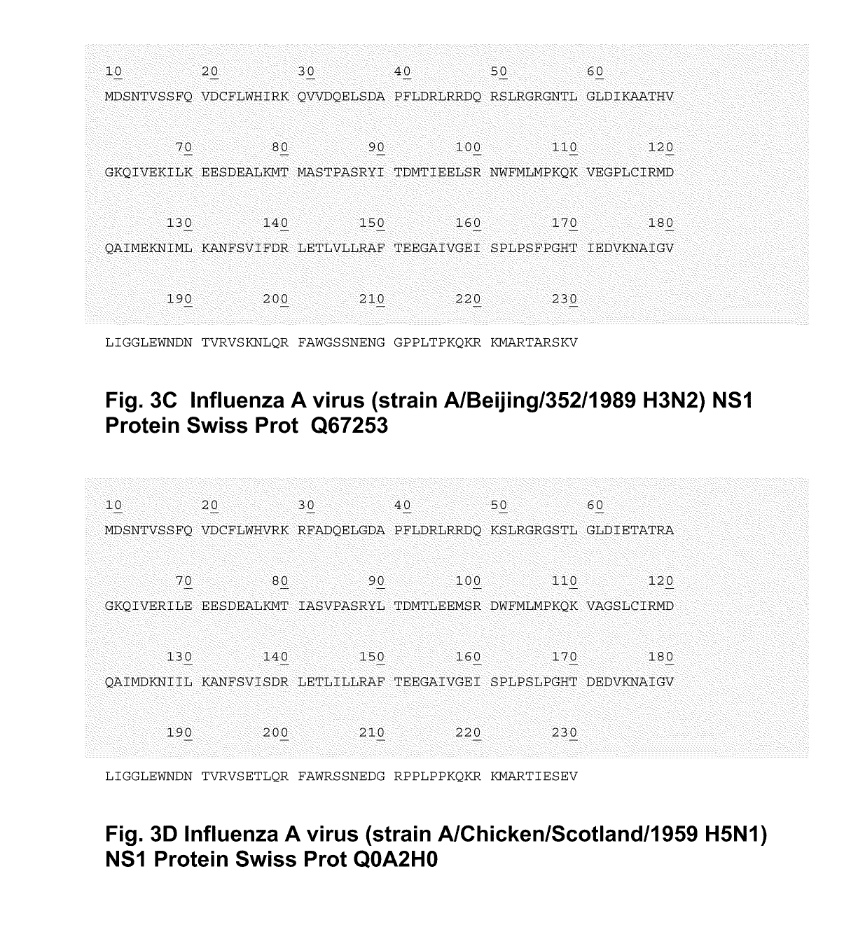

FIGS. 3A-D shows the amino acid sequence of the NS1 protein from a strain of A 2009 (H1N1) compared with exemplary NS1 sequences of H1N1, H3N2 and H5N1. Strains of A 2009 (H1N1) isolated to date have shown a strong degree of nucleic and amino acids sequence identity with one another (e.g., >99% identity within each gene). Other exemplary sequences of A 2009 (H1N1) include the following accession numbers from the Swiss Prot database: C4RSP8, C3W5W2, C4RST7, C4RSP0, C3W5S7, C3W611, C3W600, C4LNC0, C4RV09, C4RUR5, C4LQF0, C4LR02, C4RVE2, C4RVE6, C4RUB6, C4RVD8, C4RVE8, C4RVF2, C4AL64, C4AL31, C4AL73, C4ALB3, C4RSQ9. By contrast, previously isolated strains of H1N1 show less than 97% sequence identity to the genes of H1N12009. The NS1 proteins of H1N1 2009 strains isolated to date are also distinguished in that they contain 219 amino acids as distinct from 230 amino acids in the predominant extant strains of H1N1, H3N2 and H5N1 before emergence of H1N1 A2009. The omitted sequence includes the PL motif in the predominant extant strains H1N1, H3N2 and H5N1 sequences.

The A 2009 (H1N1) virus contains a combination of gene segments that previously has not been reported in swine or human influenza viruses (Garten et al, Science 325, 197-201 (2009)). The NA and M gene segments are in the Eurasian swine genetic lineage. Viruses with NA and M gene segments in this lineage were originally derived from a wholly avian influenza virus and thought to have entered the Eurasian swine population in 1979. The HA, NP, and NS gene segments are in the classical swine lineage. Viruses that seeded this lineage are thought to have entered swine around 1918 and subsequently circulated in classical swine viruses and triple reassortant swine viruses. The PB2 and PA gene segments are in the swine triple reassortant lineage Viruses that seeded this lineage, originally of avian origin, entered swine in North America around 1998. Finally, the PB1 gene segment is in the swine triple reassortant lineage. This lineage of PB1 was seeded in swine from humans at the time of the North American swine triple reassortment events and was itself seeded from birds around 1968.

Reference to the A 2009 (H1N1) virus includes any of the isolates so characterized in Genbank, Swiss-Prot database and the like, and/or isolates having a genome exhibiting at least 99% sequence identity therewith, particularly in the NS1 protein, and/or isolates having the characteristic combination of gene segments indicated above.

### III. PDZ Domains for Detection of Influenza A

Table 1 below lists the PL regions of the NS1 proteins of influenza A subtypes H5N1, H1N1 and H3N2 in predominant strains prior to the emergence of H1N1 A2009. H5N1 is the most clinically relevant subtype of pathogenic strains. H1N1 and H3N2 are the most clinically relevant subtypes of seasonal influenza A. The table also indicates whether various PDZ domains bind to the indicated PL. The table can be used to select PDZ domains for differential detection of these subtypes influenza A. For example, a PSD95 domain is useful for detecting H5N1, and INADL domain 8 is useful for detecting H1N1 and H3N2 subtypes. The PSD95 domain can be any of PDZ domains 1, 2, and 3 of PSD95, or combinations thereof. A preferred detection reagent is a protein formed from three copies of domain 2 of PSD95 in a PSD95. That is, three tandem copies interspersed by segments of PSD95 flanking its PDZ domains. In such a protein two of the copies of domain 2 of PSD95 effectively replace natural domains 1 and 3 of PSD95. Another preferred detection reagent is a protein containing PDZ domains 1, 2 and 3 of PSD95.

TABLE 1

| Influenza A | | PSD-95 | PSD95 | | |
|---|---|---|---|---|---|
| subtypes | PL | D2 | D1, D2, D3 | INADL d8 | Anti-NS1 |
| H5N1 | ESEV | ++ | ++ | − | + |
| H1N1 | RSEV | + | +− | ++ | + |
| H3N2 | RSKV | − | − | ++ | + |
| A 2009 (H1N1) | None | − | − | − | + |

Assay conditions such as buffer and temperature can be used to modulate binding to favor detection of a particular strain or differentiation among the different strains. The symbols used in the table mean as follows: ++ relatively strong binding, + detectable but relatively weak binding, +/− detectable but relatively weak binding or undetectable binding, − undetectable binding. Detectable binding means that the signal from binding is greater in a sample containing NS1 of the indicated subtype relative to a control lacking the NS1 of the indicated subtype to a significant extent taking into account random variation due to experimental error. Undetectable binding means that the signal from binding to a sample containing NS1 of the indicated subtype is within the margin of error from the signal in a control lacking NS1 of the indicated subtype.

A preferred format for subtyping influenza A uses a pan-specific binding agent (e.g., an antibody) to the NS1 protein, a PDZ polypeptide from PDS95 as shown in the table in combination with an INADL PDZ domain 8 polypeptide. The pan-specific binding agent binds to all of the indicated subtypes of influenza A. As a general rule, detectable binding of the PSD95 domain without binding of the INADL domain or significantly stronger (i.e., stronger beyond experimental error) binding of the PSD95 domain that that of the INADL domain is an indication that the influenza A subtype is H5N1 (pathogenic). Conversely, detectable binding of the INADL domain to the sample without detectable binding of the PSD95 domain to the sample or significantly stronger binding of the INADL domain to the sample than of the PSD95 to the sample is an indication that the sample contains an influenza A subtype H1N1 or H3N2 (both seasonal influenza). Detectable but weak binding of PSD95 domain 2 to the sample compared with undetectable binding distinguishes H1N1 from H3N2 as indicated in the table. Detectable but relatively weak binding of PSD95 domains 1, 2 and 3 to a sample compared with binding of INADL to the sample is also an indication that the subtype is H1N1. Lack of detectable binding of both the PSD95 polypeptide and the INADL d8 polypeptide in combination of detectable binding of the pan-specific binding agent provides an indication that the sample contains influenza A of a subtype lacking a PL motif, such as A 2009 (H1N1).

### IV. Antibodies for Detection of Influenza A

WO 08/094,525 describes pan-specific antibodies for detection of influenza A or influenza B. A pan-specific antibody for influenza A specifically binds to the NS1 protein from at least two, three or all of H1N1 (strains prior to 2009), H3N2, H5N1 and H1N1. A pan-specific antibody preferably binds to multiple strains within a subtype, for example, to H1N1 strains including a PL motif and to A 2009 (H1N1). Likewise a pan-specific antibody for influenza B specifically binds to the NS1 protein from at least 2, 3, 5 or all or substantially all known strains of influenza B. Pan-specific antibodies can be monoclonal or polyclonal.

Pan-specific antibodies can be defined by reference to either a numerically defined epitope or by a competition group defined by reference to an exemplary antibody. For influenza A, pan-specific antibodies preferably specifically bind to an epitope within residues 8-21, 9-20, 29-38 or 45-49 of FIG. 1. The X′s in this sequence can be any amino acid but are preferably an amino acid occupying the corresponding position in an NS1 protein from a strain of influenza, and more preferably the consensus amino acid occupying the corresponding position from at least two or preferably all known strains of influenza A. A consensus sequence of influenza A NS1 protein is provided in FIG. 2. Some pan-specific antibodies specifically bind to an epitope within residues 9-11 or 13-16 of FIG. 1.

Pan-specific antibodies can also be defined by a competition group; the antibodies within a competition group compete with one another for specific binding to the same antigen (i.e., an NS1 protein of influenza A).

The antibodies used can be nonhuman, humanized, chimeric, veneered, or human. Use of such antibodies is advantageous in avoiding false positives or negatives due to the presence of HAMA or heterophilic antibodies in the sample (U.S. Pat. No. 6,680,209). Humanized, chimeric or veneered versions of the antibodies listed in the tables above are preferred. Such antibodies can also be used as pharmaceutical agents in treatment of influenza A or B. Antibodies can be made from antigen-containing fragments of the protein by standard procedures according to the type of antibody (see, e.g., Kohler, et al., Nature, 256:495, (1975); and Harlow & Lane, Antibodies, A Laboratory Manual (C.S.H.P., NY, 1988) Queen et al., Proc. Natl. Acad. Sci. USA 86:10029-10033 (1989) and WO 90/07861; Dower et al., WO 91/17271 and McCafferty et al., WO 92/01047 (each of which is incorporated by reference for all purposes).

Immunization can be biased to generate pan-specific antibodies by immunizing with multiple strains of influenza A, or by immunizing with one strain and boosting with another. Alternatively, one can use a fragment from a highly conserved region of influenza A NS1 protein (e.g., 8-21, 9-20, 29-38 or 45-49 or at least three contiguous amino acids of any of these as the immunogen). Conversely, to generate a monospecific antibody, immunization with NS1 of a single strain, or a fragment of NS1 from a nonconserved region (e.g., a PL peptide of influenza A) is preferred.

The term “antibody” or “immunoglobulin” is used to include intact antibodies and binding fragments thereof. Typically, fragments compete with the intact antibody from which they were derived for specific binding to an antigen fragment including separate heavy chains, light chains Fab, Fab′ F(ab′)2, Fabc, and Fv. Fragments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes bispecific antibody.

Unless otherwise indicated the antibodies described in the present application are mouse antibodies produced from hybridomas.

### V. Other Binding Agents

Although pan-specific antibodies are preferred for use in detecting influenza A proteins, any binding agent with specific affinity for NS1 or other protein of influenza A can be used as an antibody surrogate. Surrogates includes peptides from randomized phage display libraries screened against NS1 from influenza A. Surrogates also include aptamers. Aptamers are RNA or DNA molecules selected in vitro from vast populations of random sequence that recognize specific ligands by forming binding pockets. Allosteric ribozymes are RNA enzymes whose activity is modulated by the binding of an effector molecule to an aptamer domain, which is located apart from the active site. These RNAs act as precision molecular switches that are controlled by the presence or absence of a specific effector. Aptamers can bind to nucleic acids, proteins, and even entire organisms. Aptamers are different from antibodies, yet they mimic properties of antibodies in a variety of diagnostic formats. Thus, aptamers can be used as a surrogate for pan-specific antibodies.

Likewise, although PDZ domains are preferred binding agents for detecting PL regions of NS1, an antibody specifically binding to a PL region of a particular NS1 protein of influenza A can be used as a surrogate for a PDZ domain specifically binding to that region. For example, an antibody binding to an epitope within residues 219-230 of the NS1 sequences shown in FIG. 2 or 3 is suitable.

### VI. Analyzing Sample for Influenza A

The present methods can detect with influenza A is present in a sample and further characterized the strain of influenza A with respect to presence or absence of a PL motif and, optionally if a PL motif is present, characterize the subtype (e.g., as H1N1, H3N2 or H5N1). Presence of influenza A is determined using a binding agent pan-specific for influenza A, typically for the NS1 protein. Preferably the assay is performed in a sandwich format using two pan-specific antibodies for NS1 as capture and reporter antibodies. If the antibodies are monoclonal the two antibodies should have different epitope specificities, such that the two antibodies can bind to NS1 protein simultaneously. If a pan-specific antibody is polyclonal, the same antibody can be used as capture and reporter agent because different antibodies in the polyclonal mixture bind NS1 at different epitopes. One preferred combination of antibodies for detecting influenza A is F64 3H3 (or antibody that competes therewith) as the capture antibody, and F80 3D5 (or an antibody that competes therewith) as the detection antibody. Another preferred combination is F68 4H9 (or an antibody that competes therewith) as the capture antibody and F68 8E6 (or an antibody that competes therewith) as the detection antibody.

Characterizing the strain of influenza with respect to presence or absence of a PL motif is performed using a binding agent that specifically binds to an influenza A NS1 PL motif, e.g., a PDZ polypeptide or antibody. Lack of detectable binding of this binding agent to the sample together with detectable binding of the pan-specific binding agent provides an indication that the influenza A virus present in the sample lacks a PL motif. Because the PL motif varies between different subtypes of influenza, it is preferable to include at least two different binding agent for different PL motifs. A PSD95 PDZ polypeptide and an INADL domain 8 PDZ polypeptide are particularly suitable to detect the PL motifs in most extant strains of influenza A before the emergence of A 2009 (H1N1). The assays with the binding agents for PL motif are preferably also performed in a sandwich format. The binding agents thus described can serve as the capture agents with a pan-specific binding agent as the detection agent. The pan-specific binding agent used in such methods can be the same pan-specific binding agent used as the detection agent for the pan-specific capture agent. Thus, the same detection agent can be used for one or more PDZ polypeptides and an antibody capture agents on the strip. The format of the sandwich assay can also be reversed with a pan-specific binding agent serving as the capture agent and PL motif specific agent(s) and a pan-specific binding agent as the detection agents.

Lack of detectable binding of all binding agents for different PL motifs in combination with presence of detectable binding of the pan-specific binding agent for influenza A indicates presence of an influenza A virus lacking a PL motif. Conversely, presence of detectable binding of one or more of the binding agents for different PL motifs in combination with presence of detectable binding of the pan-specific binding agent for influenza A indicates presence of an influenza A virus including a PL motif. The pattern of binding of different PL motif specific binding agents, if used, can then further indicate the subtype of influenza A present as described above. Lack of detectable binding for the different PL motifs and the pan-specific binding agent for influenza A indicates the sample lacks influenza A. The methods can be employed in individual samples or a plurality of different samples from populations of individuals. Within a population of samples, individual outcomes representing any of the outcomes or permutations thereof may occur. For example, one sample may have a pattern indicating influenza A virus, lacking a PL motif, another sample may have a pattern indicating influenza A virus with a PL motif, and another sample may lack influenza A. Sometimes most samples lack influenza A with an occasional sample indicating influenza A with a PL motif and/or a sample indicating influenza A without a PL motif.

One suitable format for combining the assays is to attach PDZ domain(s) for use in differential analysis to different regions of the same solid phase as an antibody capture reagent for use in non-subtype specific analysis. Binding of a PDZ domain to an NS1 protein in the sample can be detected using a pan-specific detection antibody. The pan-specific detection antibody used to detect binding of the PDZ domain to the NS1 protein can be the same or different as the pan-specific antibody used for non-subtype specific analysis. Thus, in a preferred format, a PSD95 domain, an INADL domain 8 and a pan-specific capture antibody for influenza A are attached to different regions of a support, and a common pan-specific detection antibody (binding to a different epitope than the pan-specific capture antibody) is used to detect binding of each of the capture reagents to an influenza A NS1 protein if present in the sample, as discussed above. Some example of preferred antibodies for use as detection agents with PDZ polypeptides include pan-specific antibody F68 8E6 (or an antibody that competes therewith) or F68 4B2 (or an antibody that competes therewith) as the detection antibody.

The present methods of analyzing influenza A can be combined with detection of influenza type B as described in e.g., WO 08/048,276. For example, the assay can include first and second pan-specific antibodies to the NS1 protein of influenza B in analogous fashion to the assays described for detecting the NS1 protein of influenza A, as described above. Such methods are performed using at least two pan-specific antibodies to the NS1 protein of influenza B binding to different epitopes. The two pan-specific antibodies bind different epitopes defined numerically as described above or can be selected from different competition groups. Detection is preferably performed using a sandwich or lateral flow format as described in more detail below.

Having analyzed the sample the results of the analysis (e.g., presence or absence of an influenza virus lacking a PL, such as A 2009 (H1N1), can be communicated to the subject, providing the sample being analyzed, treating physician and/or government agency responsible for tracking outbreaks of influenza (e.g. CDC). The information is useful for detecting emerging strains having a different status with respect to presence or absence of a PL than extant strains and subsequently tracking the emerging strains. For example, subjects infected with an emerging strain of influenza for which immunity is not widespread and for which vaccines are not yet available (as is the case for A 2009 (H1N1) subjects can be subjected to quarantining to reduce spread of the newly emergent strain and treated with one or more anti-viral compounds to reduce duration of infection and opportunity for spreading to others.

The present methods have the advantage of being rapid (e.g., results in less than 40 minutes) and field-deployable. However, if desired, the results of the present methods can be confirmed by more laborious methods performed in a laboratory, e.g., full length sequence analysis of the NS1 protein or other gene. Such analysis can readily confirm the absence of a PL motif and reveal the lineage of a new isolate from its component parts as described above for the A 2009 (H1N1) strain.

### VI. Methods of Determining Drug Resistance

The invention further provides methods of screening samples containing influenza A to determine whether the strain of influenza is sensitive or resistant to one or more drugs known to be effective in treating influenza A (e.g., tamiflu, relenza, amantadine or rimantadine). The methods can be performed on samples known to be infected with influenza A or can be combined with method described above for detecting presence and/or type and/or strain of influenza A. Such methods determine whether a drug binds to a sample known or suspected of containing influenza A. The presence or extent of binding provides an indication whether influenza A present in the drug is sensitive or resistant with present or relatively greater extent of binding being indicative of sensitivity. The binding of the drug to the sample can be compared with various negative or positive controls. A preferred control is a binding reagent for an influenza A protein. The binding reagent may or may not be specific for the same protein of influenza A as the drug under test binds. For example, the binding agent can be a pan-specific antibody to NS1. In such case, detectable binding of the binding agent and of the drug indicates the sample contains influenza A that is sensitive to the drug. Conversely, detectable binding of the binding agent to the sample and lack of detectable binding of the drug to the sample indicates the sample contains influenza A that is resistant to the drug. The assay can also be configured to relate relative binding of the binding agent and drug to extent of drug resistance. For example, the relative binding of the binding agent and drug can be measured on a series of strains of known extent of drug resistance to set up a scale associating relative binding with extent of drug resistance. The relative binding of the binding agent and drug to a sample under test can then be assigned an extent of drug resistance on the scale. Alternatively or additionally, comparable samples of influenza A of known drug resistance or sensitivity can be assayed in parallel with a sample under a test and the extent of resistance or sensitivity of the sample under test interpolated from such contemporaneous controls.

The drug screening assay is preferably performed in a format in which the drug and binding agent are immobilized or immobilizable (i.e., become immobilized in the course of the assay). The format is preferably a sandwich assay in which the drug and binding agent serve as capture agents. The same or different reporter agents can be used for the drug and binding agent depending in part whether the drug and binding agent bind to the same protein of influenza. For example, one suitable format uses a pan-specific anti-NS1 antibody, and one or more anti-influenza drugs as the capture agents, a pan-specific anti-NS1 antibody as reporter for the capture anti-NS1 antibody and a pan-specific antibody to neuraminidase if the drug is tamiflu or relenza or to M2 protein if the drug is amantadine or rimantadine. The above methods are amenable to testing any number of drugs simultaneously, for example, by immobilizing multiple drugs to different regions of a support. At least one reporter agent is needed for each different protein bound by drugs under test.

The same principles and strategy can be used in testing anti-viral drugs for sensitivity or resistance on influenza B or C, or in testing anti-viral drugs on other viruses (e.g., HIV or rhinovirus). The binding agent for detecting presence of the virus should be appropriately selected to bind to a viral protein of the virus under test as should the detection agents. Drugs for which the viral target is indicated to be sensitive by such tests are thus indicated as suitable for administration to a patient infected with the virus.

A variety of formats are available for immobilizing a drug. In some method the drug is striped directly on the membrane. In other methods, the drug is conjugated to a carrier that can bind the membrane, optionally via covalently bonding. For example, the drug can be conjugated to biotin and bound to streptavidin coated on a membrane support. In another example, the drug is conjugated to a protein such as bovine serum albumin, which binds to a membrane support. Optionally, a spacer can be placed between the bovine serum albumin and the protein. In other methods, multiple drug molecules are attached to a polymer, which is in turn striped onto a membrane. In other methods, the drug is attached to a label such as europium beads and is used as a detection agent with an antibody serving as a capture agent.

In a further variation, beads (e.g., sepharose or agarose) are functionally with a drug, optionally with a spacer between the drug and the bead. A sample is then split into two parts. One part is pre-treated with beads coated with the drug to deplete the viral target protein of the drug from the sample (assuming the viral target is sensitive to the drug). The other part is not pretreated with the beads. Each of the two parts is then analyzed separately for present of the viral target. The test system can be a simple antibody capture and antibody detection system. If both split samples are positive, then the sample contains a drug resistant strain (i.e., contact with the beads does not deplete). If the sample pretreated with the bead-molecule is negative and the untreated sample is positive, then the strain is sensitive to the drug. The relative amount of viral target in the samples can provide an indication of the extent of viral sensitivity by interpolation from resistant and sensitive control samples or a scale of relating drug sensitivity to ratio of viral target derived from analysis of such samples.

### VII. Detection Formats

The methods of analyzing influenza A can be performed in a variety of different formats including immunoprecipitation, Western blotting, ELISA, radioimmunoassay, competitive and immunometric assays. See Harlow & Lane, Antibodies, A Laboratory Manual (CSHP NY, 1988); U.S. Pat. Nos. 3,791,932; 3,839,153; 3,850,752; 3,879,262; 4,034,074, 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876. Direct immunofluorescence assays and multiplexed assays may also be used. See Chen et al., Clinical Chemistry 45:1693-1694, 1999; Oliver et al., Clinical Chemistry 44:2057-2060, 1998; Fulton et al., Clinical Chemistry 43:1749-1756, 1997. Examples of multiplexed immunoassays include, e.g., BD™ Cytometric Bead Array (CBA) (BD Biosciences, San Jose, Calif.).

Immunometric or sandwich assays are a preferred format (see U.S. Pat. Nos. 4,376,110, 4,486,530, 5,914,241, and 5,965,375). Such assays use one antibody or population of antibodies or a PDZ domain immobilized to a solid phase as a capture agent, and another antibody or population of antibodies or a PDZ domain in solution as detection agent. Typically, the detection agent is labeled. If an antibody population is used, the population typically contains antibodies binding to different epitope specificities within the target antigen. Accordingly, the same population can be used for both capture agent and detector agent. If monoclonal antibodies are used as detection and detection agents, first and second monoclonal antibodies having different binding specificities are used for the solid and solution phase. Capture and detection agents can be contacted with target antigen in either order or simultaneously. If the capture agent is contacted first, the assay is referred to as being a forward assay. Conversely, if the detection agent is contacted first, the assay is referred to as being a reverse assay. If target is contacted with both capture agent and detection agent simultaneously, the assay is referred to as a simultaneous assay. After contacting the sample with capture and detection antibodies, a sample is incubated for a period that usually varies from about 10 min to about 24 hr and is usually about 1 hr. A wash step can then be performed to remove components of the sample not specifically bound to the detection agent. When capture and detection agents are bound in separate steps, a wash can be performed after either or both binding steps. After washing, binding is quantified, typically by detecting label linked to the solid phase through binding of labeled solution antibody. Usually for a given pair of capture and detection agents and given reaction conditions, a calibration curve is prepared from samples containing known concentrations of target antigen. Concentrations of antigen in samples being tested are then read by interpolation from the calibration curve. Analyte can be measured either from the amount of labeled solution antibody bound at equilibrium or by kinetic measurements of bound labeled solution antibody at a series of time points before equilibrium is reached. The slope of such a curve is a measure of the concentration of target in a sample.

Competitive assays can also be used. In some methods, target antigen in a sample competes with exogenously supplied labeled target antigen for binding to an antibody or PDZ detection reagent. The amount of labeled target antigen bound to the detection reagent is inversely proportional to the amount of target antigen in the sample. The detection reagent can be immobilized to facilitate separation of the bound complex from the sample prior to detection (heterogeneous assays) or separation may be unnecessary as practiced in homogeneous assay formats. In other methods, the detection reagent is labeled. When the detection reagent is labeled, its binding sites compete for binding to the target antigen in the sample and an exogenously supplied form of the target antigen that can be, for example, the target antigen immobilized on a solid phase. Labeled detection reagent can also be used to detect antibodies in a sample that bind to the same target antigen as the labeled detection reagent in yet another competitive format. In each of the above formats, the detection reagent is present in limiting amounts roughly at the same concentration as the target that is being assayed.

Lateral flow devices are a preferred format. Similar to a home pregnancy test, lateral flow devices work by applying fluid to a test strip that has been treated with specific biologicals. The lateral flow typically contains a solid support (for example nitrocellulose membrane), a sample addition area, and a read-out area that contains one or more zones or lines containing immobilized antibody or PDZ polypeptide agents. The lateral device may also contain an area containing one or more labeled detection agents (e.g., antibodies to NS1 or PDZ polypeptides) that mix with the sample as it diffuses along the strip before it reaches the read out area. Alternatively, the sample can be combined with one or more detection agents before being applied to the strip. The presence of an analyte is signaled by a visible or readable (depending on the label) line on the support. The lateral flow can also include positive and negative controls, such as recombinant NS1 protein or a goat anti-mouse antibody. Methods and devices for lateral flow separation, detection, and quantification are described by, e.g., U.S. Pat. Nos. 5,569,608; 6,297,020; and 6,403,383 incorporated herein by reference in their entirety.

Multiplexed assays such as a multiplexed bead assay can be used. A multiplexed bead assay, such as, e.g., the BD Cytometric Bead Array, is a series of spectrally discrete particles that can be used to capture and quantitate soluble analytes. The beads can bear different binding agents such as the PDZ, pan-specific or any other binding agents discussed herein. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

Suitable detectable labels for use in the above methods include any moiety that is detectable by spectroscopic, photochemical, biochemical, immunochemical, electrical, optical, chemical, or other means. For example, suitable labels include biotin for staining with labeled streptavidin conjugate, fluorescent dyes (e.g., fluorescein, Texas red, rhodamine, green fluorescent protein, and the like), radiolabels (e.g., ³H, ¹²⁵I, ³⁵S, ¹⁴C, or ³²P), enzymes (e.g., horseradish peroxidase, alkaline phosphatase and others commonly used in an ELISA), and colorimetric labels such as colloidal gold or colored glass or plastic (e.g., polystyrene, polypropylene, latex beads). Patents that described the use of such labels include U.S. Pat. Nos. 3,817,837; 3,850,752; 3,939,350; 3,996,345; 4,277,437; 4,275,149; and 4,366,241. See also Handbook of Fluorescent Probes and Research Chemicals (6th Ed., Molecular Probes, Inc., Eugene Oreg.). Radiolabels can be detected using photographic film or scintillation counters, fluorescent markers can be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The level of influenza NS1 protein in a sample can be quantified and/or compared to controls. Suitable negative control samples are e.g. obtained from individuals known to be healthy, e.g., individuals known not to have an influenza viral infection. Specificity controls may be collected from individuals having known influenza type, subtype and/or strain, or individuals infected with viruses other than influenza. Control samples can be from individuals genetically related to the subject being tested, but can also be from genetically unrelated individuals. A suitable negative control sample can also be a sample collected from an individual at an earlier stage of infection, i.e., a time point earlier than the time point at which the test sample is taken. Recombinant NS1 of influenza A and optionally influenza B or an anti-mouse isotype antibody can be used as a positive control.

Western blots show that NS1 levels in biological samples are sufficient to allow detection of these antigens in a variety of different possible immunoassay formats. However, should the levels of NS1 in a particular biological sample prove to be limiting for detection in a particular immunoassay format, then, the live virus in a biological sample can be amplified by infecting cells in vitro, i.e., the NS1 protein in the virus-amplified sample should be detectable in about 6 hr to about 12 hr. The yield of NS1 antigen in biological samples and virus-amplified samples can also be improved by inclusion of protease inhibitors and proteasome inhibitors.

### VIII. Samples

Any biological sample from a subject can be used that contains or is thought might contain a detectable concentration of influenza proteins and preferably of NS1. For example, samples are often obtain from humans having or suspected of or at elevated risk of having influenza (e.g., through contact with others having influenza). Examples of samples that can be used are lung exudates, cell extracts (respiratory, epithelial lining nose), blood, mucous, and nasal swabs, for example. A high concentration of NS1 can be found in nasal swabs. Thus, a preferred sample for identification of NS1 is nasal secretion.

The methods can be used be used without any preconceptions of the type or strain of influenza A present in a sample or can be used when it is known or suspected that a new strain of influenza A has emerged and that strain is lacking a PL motif in its NS1 protein, as is the case for A 2009 (H1N1). In such cases, the methods can be used to test subjects believed to be at enhanced risk of the emergent strain for example, subjects having been in contact with others infected with the new strain. Such individual may include subjects who have co-occupied a room or vehicle with an infected subject.

Binding of NS1 to an antibody occurs in the presence of up to 0.05% SDS, including 0.03% and 0.01%. Therefore, when the nasal or other bodily secretion is not likely to easily be used in a lateral flow format, it can be treated with SDS. Preferably, the amount of SDS added is up to a final concentration of 0.01%, more preferably 0.03% and even more preferably, 0.05%.

### IX. Diagnostic and Therapeutic Kits

Kits are provided for carrying out the present methods. Some kits include a pan-specific antibody that specifically binds NS1 protein of influenza A and one or more PDZ polypeptides, e.g., INADL d8 and/or PSD95. Some kits include an antibody that specifically binds a viral protein and one or more drugs that inhibit infection of that virus. For example, the antibody can be a pan-specific antibody to influenza A NS1 protein and the drug(s) can be tamiflu, relenza, amantadine or rimantadine. The kits can also include reporter agents ad described above, labels for detecting reporter reagents. The kit can also include a means, such as a device or a system, for removing the influenza viral NS1 from other potential interfering substances in the biological sample. The instant kit can further include, if desired, one or more of various components useful in conducting an assay: e.g., one or more assay containers; one or more control or calibration reagents; one or more solid phase surfaces on which to conduct the assay; or, one or more buffers, additives or detection reagents or antibodies; one or more printed instructions detailing how to use the kit to detect or type influenza A or other virus e.g., as package inserts and/or container labels, for indicating the quantities of the respective components that are to be used in performing the assay, as well as, guidelines for assessing the results of the assay. Such kits can also contain components useful for conducting a variety of different types of assay formats, including e.g. test strips, sandwich ELISA, Western blot assays, latex agglutination, direct immunofluorescence assays, multiplexed assays and the like.

All publications, patent filings and sequences associated with accession numbers or the like cited in this specification are herein incorporated by reference as if each individual publication, patent or accession number were specifically and individually indicated to be incorporated by reference. If more than one version of a sequence has been associated with the same accession number at different times, reference to a deposit number should be construed as applying to the version in existence at the effective filing date of the application dating back to a priority application if the accession number is also referenced in the priority application. Various changes may be made and equivalents may be substituted without departing from the true spirit and scope of the invention. Unless otherwise apparent from the context, any feature, step or embodiment can be used in combination with any other feature, step or embodiment.

### EXAMPLES

### Example 1

### Antibody Production

Monoclonal antibodies were prepared to specifically bind to subtype NS1 proteins (e.g., H5N1), NS1 PL classes (e.g., ESEV) and for pan-specificity (influenza A). The strategy for the generation of monoclonal antibodies to NS1 was as follows:
1. GST and MBP fusion proteins of NS1 were generated. The cloning vectors were obtained from Pharmacia (GST) or New England Biolabs (MBP). The NS1 coding regions were synthesized using overlapping oligonucleotides by DNA 2.0 (Menlo Park, Calif.).
2. Mice were immunized with MBP-NS1 fusion proteins at doses ranging from 10-100 ug per dose in CFA then IFA and PBS.
3. Splenocytes and lymphocytes were harvested 3 days after the last boost with the corresponding GST-NS1 fusion protein and fused with FOX-NY myeloma cells according to Kohler and Milstein (Nature 1975).
4. The hybridomas were screened first with MBP-NS1 in an ELISA. The positive wells were cloned and rescreened with a panel of MBP and GST NS1 and classified into pan-reactive or subtype reactive.
5. Further screenings were done using Western blots to verify the molecular weight of the target protein that is consistent with NS1.
6. An additional screening is performed to check compatibility with PDZ capture.
7. Steps 5 and 6 were repeated with eukaryotic expressed NS1 in the form of a cell lysate.
8. The antibodies were checked for compatibility with a lateral flow format.
9. The antibodies were checked for the ability to detect NS1 in a clinical specimen.

### Example 2

### Lateral Flow

Recombinant PDZ domain proteins or antibodies are deposited on RF120 Millipore membrane using a striper. For example, the PDZ proteins PSD95D1-3, INADL D8 and one or more pan-specific antibodies NS1 are deposited at a concentration of 0.5 mg/ml for PDZ polypeptides or about ˜0.7 mg/ml for antibody or mixture of antibodies. A control band of goat anti-mouse antibody (GAM) at 0.5 mg/ml is also deposited. A sample containing NS1 protein is combined with gold conjugated monoclonal anti-NS1 such as 4B2 in 100 ul volume in Tris buffered saline-Twen (TBS-T buffer, Millipore). Human nasal aspirates are diluted and stored in saline or M4 solution (Remel, Inc, Lenexa, Kans.), as indicated.

The steps are set out below.

1. Prepare cards with a sample membrane and sink pad.

2. Stripe membrane with the PDZ polypeptides and/or antibodies (see above for conc.)

3. Dry the membrane overnight at 56 degrees, then cut the cards into strips 4.26 mm wide.

4. Attach a glass fiber sample pad to the bottom of the strip and place the entire strip inside a cassette for testing.

5. Thaw sample to be tested and add 80 μl of sample to 20 μl of buffer. Pipette up and down several times to mix.

6. Spike 8 μl of the gold-conjugated (Au-) detector mix into the sample/buffer solution. This detector mix is 4 μl of Au-F68-4B2 with 4 μl of Au-F68-3D5. Pipette up and down several times to mix.

7. Add 100 μl of the prepared sample to the sample well on the cassette.

8. Read the test and control lines on the cassette at 15 minutes post-addition of sample. The control line (goat anti-mouse antibody) should be clearly visible for any test results to be read reliably.

Alternatively, the cards are prepared as in the liquid gold protocol except the sample pad is affixed to the card before striping. When the captures were striped down, the gold-conjugated detector mix (which here also contained a conjugate diluent) was sprayed on the sample pad at the base of the card. The cards were dried, cut, and placed in cassettes as with the liquid test. When the human samples are prepared, they were treated with only the buffer solution before 100 μl was run on the cassette (no additional gold-conjugated detector mix was added).

Additional examples of methods for isolating antibodies to NS1 protein, immobilizing such antibodies and PDZ polypeptides on a strip and detecting influenza NS1 protein by lateral flow assay have been described in WO 08/094,525, WO 08/048,276 and WO 07/018,843.

### Example 3

### PDZ Proteomics Identify Swine Flu

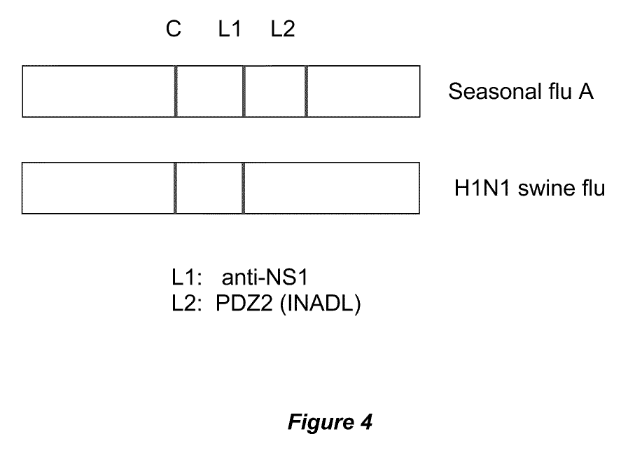

FIG. 4 is a schematic lateral flow assay to identify Swine flu. Three binding reagents are deposited on the strip: goat anti-mouse antibody (C) (positive control), pan-specific anti-NS1 antibody (L1) and INADL domain 8(L2) A pattern containing three bands indicates presence of seasonal flu A, whereas presence of the C and L1 stripes and not the L2 bands indicates swine flu.

### Example 4

### PDZ Profiler for Flu Subtyping

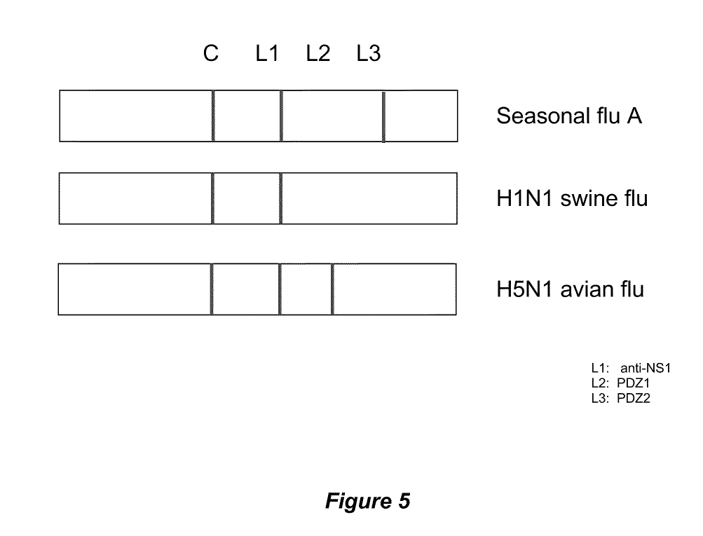

FIG. 5 illustrates expansion of the previous assay by including two PDZ polypeptides (L2 and L3) to distinguish seasonal flu A and H5N1 avian flu as well as identifying swine flu. PSD95 is suitable for use as the L2 PDZ polypeptide and INADL d8 as the L3 PDZ polypeptide.

### Example 5

### Drug Resistance Profiler for Flu A

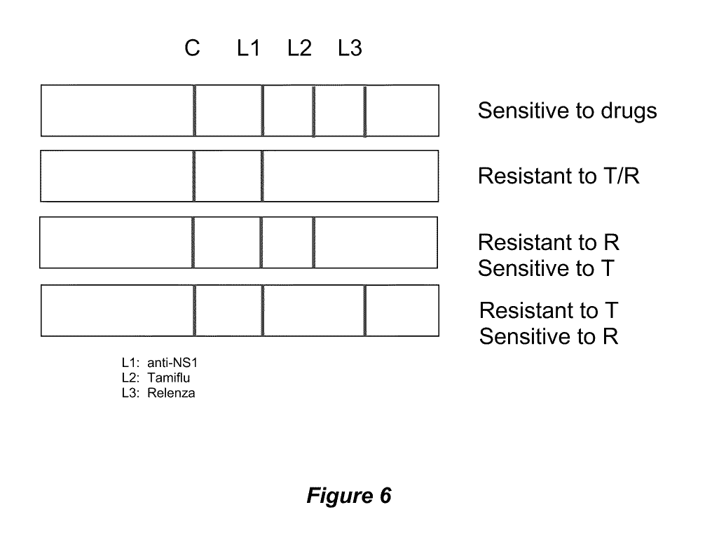

FIG. 6 is a schematic illustrating use of strip test to type influenza A for drug resistance. Two common influenza drugs, tamiflu and Relenza are deposited on a strip together with goat anti-mouse antibody (C) as a positive control, and a pan-specific antibody for NS1 (L1) to indicate presence of influenza A. Bands for L2 and L3 indicate sensitivity to these drugs.

1. A method of analyzing a sample for influenza A infection, comprising
contacting a sample with a pan-specific binding reagent for an influenza A protein and with a PL-motif binding reagent;
determining binding of the pan-specific binding reagent and the PL-motif binding agent to the sample;
analyzing the sample from comparative binding of the pan-specific binding reagent and the PL-motif binding agent, wherein
(a) detectable binding of the pan-specific binding agent and lack of detectable binding of the PL-motif binding agent provides an indication the sample contains a strain of influenza A lacking a PL-motif;
(b) detectable binding of the pan-specific binding agent and the PL-motif binding agent provides an indication the sample contains a strain of influenza A containing a PL motif; and
(c) lack of detectable binding of both the pan-specific binding agent and the PL-motif binding agent provides an indication the sample is not infected with influenza A.

2. The method of claim 1, wherein the determining step indicates detectable binding of the pan-specific binding agent and lack of detectable binding of the PL-motif binding agent provides an indication the sample contains a strain of influenza A lacking a PL motif.

3. The method of claim 2 further comprising informing a subject from whom the sample was obtained or a physician treating the patient that the method has provided an indication the sample contains a strain of influenza A lacking a PL motif.

4. The method of claim 2, further comprising subjecting the subject from whom the sample was obtained to a new treatment regime or quarantine regime.

5. The method of claim 1, wherein the pan-specific binding reagent is an antibody that specifically binds to an influenza A NS1 protein.

6. The method of claim 1, wherein the pan-specific binding reagent is a monoclonal antibody that specifically binds to an influenza A NS1 protein.

7. The method of claim 1, wherein the PL motif binding reagent is a first PDZ polypeptide and the contacting step further comprises contacting the sample with a second PDZ polypeptide, and the determining step further comprises determining binding of the second PDZ polypeptide to the sample; wherein greater binding of the pan-specific binding reagent than either the first or second PDZ polypeptides provides an indication that the sample contains a strain of influenza A lacking a PL motif.

8. The method of claim 7, wherein
(a) detectable binding of the pan-specific binding agent and lack of detectable binding of the first and second PL-motif binding agents provides an indication the sample contains a strain of influenza A lacking a PL motif;
(b) detectable binding of the pan-specific binding agent and either or both of the PL-motif binding agent provides an indication the sample contains a strain of influenza A containing a PL motif; and
(c) lack of detectable binding of both the pan-specific binding agent and the PL-motif binding agents provides an indication the sample is not infected with influenza A.

9. The method of claim 7, wherein the determining step indicates detectable binding of the pan-specific binding agent and lack of detectable binding of the first and second PL-motif binding agents and provides an indication the sample contains a strain of influenza A lacking a PL motif.

10. The method of claim 7, wherein the pan-specific binding reagent is an antibody that specifically binds to an influenza A NS1 protein, the first PDZ polypeptide comprises INADL domain 8 and the second PDZ polypeptide comprises a PSD95 PDZ domain.

11-14. (canceled)

15. The method of claim 14, wherein the pan-specific binding reagent is a polyclonal antibody to NS1, the second pan-specific binding reagent is a polyclonal antibody to NS1, and the PL-motif binding agent is a PDZ polypeptide.

16. The method of claim 13, wherein the same detection agent is used in determining binding of the pan-specific binding reagent and the PL-motif binding agent.

17. The method of claim 13, wherein the pan-specific binding agent is a monoclonal antibody to NS1, the second pan-specific binding agent is a second monoclonal antibody to NS1 binding to a different epitope that the monoclonal antibody to NS1, the PL-motif binding agent is a PDZ polypeptide, and the second monoclonal antibody is used as the detection agent in determining binding of the pan-specific binding reagent and the PL-motif binding agent.

18. The method of claim 1, wherein the sample is from an individual suspected of being infected with a strain of influenza A lacking a PL motif.

19. The method of claim 1, wherein the sample is from an individual exposed to another subject having influenza A lacking a PL motif.

20. The method of claim 1, wherein the sample is from an individual suspected of being infected with H1N1 influenza A lacking a PL motif.

21. The method of claim 20, wherein the sample is from an individual suspected of being infected with A 2009 (H1N1).

22. A method of analyzing a sample for influenza A infection, comprising
contacting a sample with a pan-specific binding reagent for an influenza A protein and with a PL-motif binding reagent;
determining binding of the pan-specific binding reagent and the PL-motif binding agent to the sample;
wherein the determining indicates detectable binding of the pan-specific binding agent and lack of detectable binding of the PL-motif binding agent and thereby provides an indication the sample contains a strain of influenza A lacking a PL motif.

23. (canceled)

24. The method of claim 23, wherein the sample is suspected of containing influenza A of H1N1 subtype lacking a PL motif.

25. The method of claim 23, wherein the sample is suspected of containing 2009 swine flu.

26-43. (canceled)

44. A method of analyzing a plurality of samples for influenza A infection, comprising
contacting the plurality of sample with a pan-specific binding reagent for an influenza A protein and with a PL-motif binding reagent;
determining binding of the pan-specific binding reagent and the PL-motif binding agent to the plurality of samples;
analyzing the samples from comparative binding of the pan-specific binding reagent and the PL-motif binding agent, wherein
(a) at least one sample shows detectable binding of the pan-specific binding agent and lack of detectable binding of the PL-motif binding agent providing an indication it contains a strain of influenza A lacking a PL-motif; and
(b) at least one sample shows lack of detectable binding of both the pan-specific binding agent and the PL-motif binding agent providing an indication the sample is not infected with influenza A.

45. The method of claim 44, further wherein at least one sample shows detectable binding of the pan-specific binding agent and the PL-motif binding agent providing an indication it contains a strain of influenza A containing a PL motif.
